(19) United States
(12) Patent Application Publication
(10) Pub. No.: US 2017/0164921 A1
(43) Pub. Date: Jun. 15, 2017

(54) RADIOGRAPHIC DEVICE

(71) Applicant: SHIMADZU CORPORATION, KYOTO-SHI (JP)

(72) Inventors: Dai HIROSE, KYOTO-SHI (JP); Koki YOSHIDA, KYOTO-SHI (JP)

(73) Assignee: SHIMADZU CORPORATION, KYOTO-SHI (JP)

(21) Appl. No.: 15/373,620

(22) Filed: Dec. 9, 2016

(30) Foreign Application Priority Data
Dec. 9, 2015 (JP) 2015-240186
Dec. 5, 2016 (JP) 2016-236031

(51) Int. Cl. A61B 6/00; A61B 6/04

(52) U.S. Cl. CPC A61B 6/54; A61B 6/0407; A61B 6/42; A61B 6/40; A61B 6/4452; A61B 6/504; A61B 6/461; A61B 6/5211

(57) ABSTRACT

An X-ray imaging apparatus that reduces the workload of a user when the X-ray imaging is performed while moving a table in 2 axes directions in a loading plane relative to an X-ray irradiation detection element. The X-ray imaging apparatus 100 comprises an X-ray irradiation detection element 1 including an X-ray irradiation element irradiates an X-ray to a subject 10 and an X-ray detection element 1 that detects the X-ray transmitting said subject 10, a table 2 that can load the subject 10 on the loading plane, and a control element 3 that moves the X-ray irradiation detection element 1 or at least one side of the table 2 in the first direction and the second direction orthogonal each other along a pathway of the relative position of the preregistered loading plane 2c and the X-ray irradiation detection element 1 when the X-ray imaging of the subject 10 is performed.

### CROSS REFERENCE TO RELATED APPLICATIONS

This application relates to and claims priority from JP Ser. No.: JP 2015-240186 filed Dec. 9, 2015 and relates to and claims priority from JP Ser, No.: 2016-236031 filed Dec. 5, 2016. The entire contents of each of which are incorporated herein by reference.

### FIGURE SELECTED FOR PUBLICATION

FIG. 1

### BACKGROUND OF THE INVENTION

Field of the Invention

The present invention relates to an X-ray imaging apparatus, and more particularly, relates to an X-ray imaging apparatus having an X-ray irradiation detection element.

Description of the Related Art

Conventionally, it is known that there is an X-ray imaging apparatus having an X-ray irradiation detection element (e.g., refer to Patent Document 1).

Patent Document 1 discloses the X-ray imaging apparatus comprising; an X-ray detection element including an X-ray tube that irradiates an X-ray to the subject and an X-ray irradiation detection element that detects the X-ray transmitting the subject; and a table that is relatively movable relative to the X-ray irradiation detection element in each orthogonal longitudinal direction and the wide direction (2 axes direction) in the loading plane in the state in which the subject is in place on the loading plane.

### RELATED PRIOR ART DOCUMENTS

### Patent Document

### Patent Document: Laid Open JP 2010-162278

### ASPECTS AND SUMMARY OF THE INVENTION

### Objects to be Solved

Here, according to the conventional X-ray imaging apparatus, the table is adjusted relative to each position in the respectively orthogonal length direction and width direction (2 axes direction) by the user's operation, and is structured to be relatively movable relative to the X-ray irradiation detection element. Despite no clear description of the X-ray imaging apparatus in Patent Document 1, supposedly it is configured as well and when the X-ray imaging is performed while relatively moving the table in the length direction and width direction relative to the X-ray irradiation detection element, the user has to operate both relative-moves as for the table in the length direction and width direction and the operational workload for the user is problematically big. Particularly, when a contrast agent is applied for imaging of blood vessel, the user has to operate the velocity of relative moving in addition to the operation of the table in the length direction and width direction relative to the X-ray irradiation detection element, so that the operational workload for the user can be bigger. Further, the X-ray imaging area is narrowed to reduce the X-ray radiation dose to be irradiated to the subject, the X-ray imaging area relative to the blood vessel becomes small, and the workload of the user tracking the contrast agent moving inside the blood vessel is further bigger.

The present invention has been proposed in order to solve the aforementioned problems, and an object of the present invention is to provide an X-ray imaging apparatus that can easily and absolutely measure a distance.

### Means for Solving the Problem

To achieve the above purpose, an X-ray imaging apparatus according to the aspect of the present invention comprises: an X-ray irradiation detection element including an X-ray irradiation element that irradiates an X-ray to a subject and an X-ray detection element that detects the X-ray transmitting said subject; a table that can load the subject on the loading plane, and a control element that moves the X-ray irradiation detection element or at least one side of the table in the first direction and the second direction orthogonal each other along a pathway of the relative position of the preregistered loading plane and the X-ray irradiation detection element when the X-ray imaging of the subject is performed.

When the X-ray imaging of the subject is performed, as set forth above, the X-ray imaging apparatus according to the aspect of the present invention moves the X-ray irradiation detection element or at least one side of the table in the first direction and the second direction orthogonal each other along the pathway of the relative position of the pre-registered loading plane and the X-ray irradiation detection element. Accordingly, the control element moves automatically the table relatively in the first direction and the second direction relative to the X-ray irradiation detection element along the pre-registered pathway, so that the user's operational workload can be reduced when an X-ray imaging is performed while relatively moving the table in the 2 axes direction (the first direction and the second direction) in the loading plane relative to the X-ray radiation detection element. As results, even when the X-ray imaging area is narrowed to suppress the X-ray dose being irradiated to the subject, the imaging target region of the subject can be automatically followed and the imaging target region of the subject can be imaged easily and adequately.

The X-ray imaging apparatus according to the aspect of the present invention further preferably comprises; a velocity operation element that receives an operation to control a velocity of relative-move of the table moving along the pathway in the first direction and the second direction relative to the X-ray irradiation detection element; wherein the control element controls the velocity of the relative-move of the table moving in the first direction and the second direction relative to the X-ray irradiation detection element based on the operation of the velocity operation element. According to such structure, the user should control only the velocity of the relative-move of the table moving in the first direction and the second direction relative to the X-ray irradiation detection element, so that the user can facilitate to track the contrast agent moving in the blood vessel when an X-ray imaging of the blood vessel is performed using the contrast agent. As results, when the X-ray imaging is performed while moving the table in the 2 axes directions in the loading plane relative to the X-ray irradiation detection element, the operation workload of the user can be reduced further.

According to the X-ray imaging apparatus according to the aspect as described above preferably, the control element is structured to acquire the pathway based on a positional data of the first direction and the second direction of the table designated by the user relative to the X-ray irradiation detection element. According to such structure, a necessary X-ray image of the subject can be acquired adequately so that the user can set the pathway every X-ray imaging.

Preferably in such case, the positional data should include data of plural representative points of the table designated by the user's operation relative to the X-ray irradiation detection element, and the control element is structured to acquire the pathway based on the data of plural representative points of the table. According to such structure, the pathway is acquired based on the representative points, so that the user can easily to set up the pathway by designating the representative points along the imaging target region of the subject.

According to such structure, the control element is preferably structured to acquire the pathway by adjusting the region between plural representative points by interpolating with the straight line or the curved line regarding the structure t to acquire the pathway based on the data of plural representative points of the table. According to such structure, the user can set up easily the pathway by interpolating with designating many representative points.

According to such structure that the above control element acquires the pathway based on the positional data of the table in the first direction and the second direction relative to the X-ray irradiation detection element designated by the user, preferably, the positional data is structured so as to include the tracking data of the table relatively moved by the user's operation relative to the X-ray irradiation detection element and the control element is structured so as to acquire the pathway based on the tracking data of the table. According to such structure, the pathway can be acquired by the user's operation based on the tracking data of the table relatively moved relative to the X-ray irradiation detection element, so that the pathway can be set by relatively easy operation.

The control element described above acquires the pathway based on a positional data of the first direction and the second direction of the table designated by the user relative to the X-ray irradiation detection element, and preferably the positional data includes data identifying the blood vessel and the control element acquires the pathway along the identified blood vessel. According to such structure, the pathway thereof can be acquired along the identified blood vessel common to each subject, so that the pathway can be further easily set up.

In such case, the positional data includes preferably the specific data to extrapolate the pathway of the blood vessels and the control element extrapolates the pathway based on the data of the specific points in addition to the data identifying the blood vessels. According to such structure, the pathway can be extrapolated based on the data of specific points in addition to the data identifying the blood vessel, so that the pathway can be easily set up regardless experiences of the user.

According to such structure in which the control element acquires the pathway based on the data of the tracking of the table, the control element registers the tracking along one direction of the length direction of the table and when the tracking of the table is returned to the other direction, the tracking is re-registered from the returned position excluding the tracking of the returned portion of the table. According to such structure, the tracking can be updated by just returning the table, so that the tracking can be easily updated.

The X-ray imaging apparatus according to the above aspect preferable further comprises a display element to display the pathway and the control element can update the pathway displayed on the display element based on the revision instruction from the user. According to such structure, the display element can display the target pathway to be updated. so that the user can easily update the tracking while watching the display element and confirming the display.

### Effect of the Invention

According to the aspect of the present invention, when the X-ray imaging is performed while moving the table in the 2 axes directions in the loading plane relative to the X-ray irradiation detection element, the operation workload of the user can be reduced.

The above and other aspects, features and advantages of the present invention will become apparent from the following description read in conjunction with the accompanying drawings, in which like reference numerals designate the same elements.

### BRIEF DESCRIPTION OF THE DRAWINGS

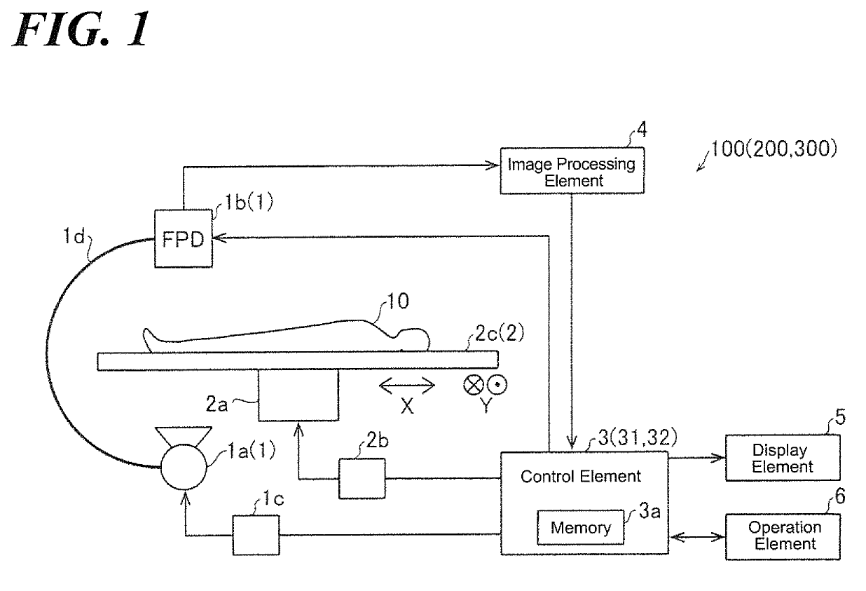

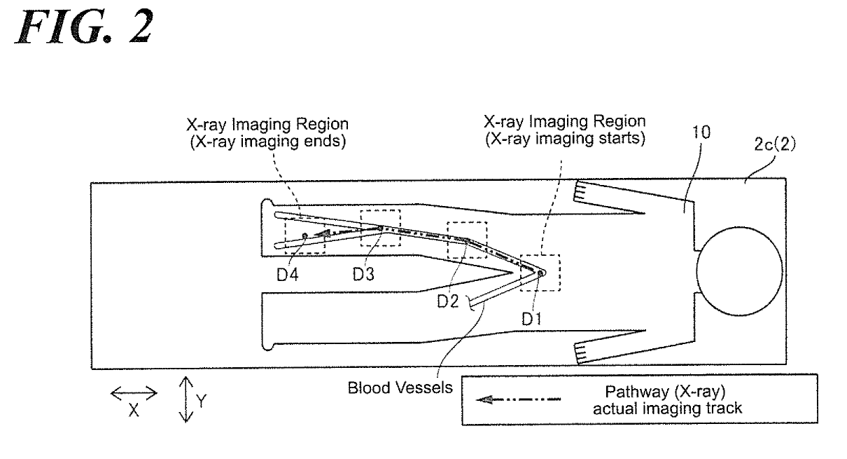

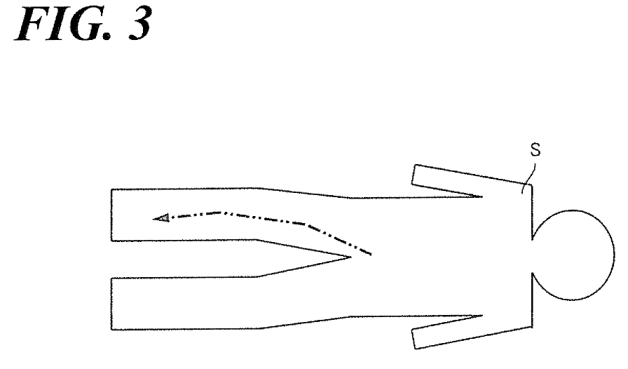

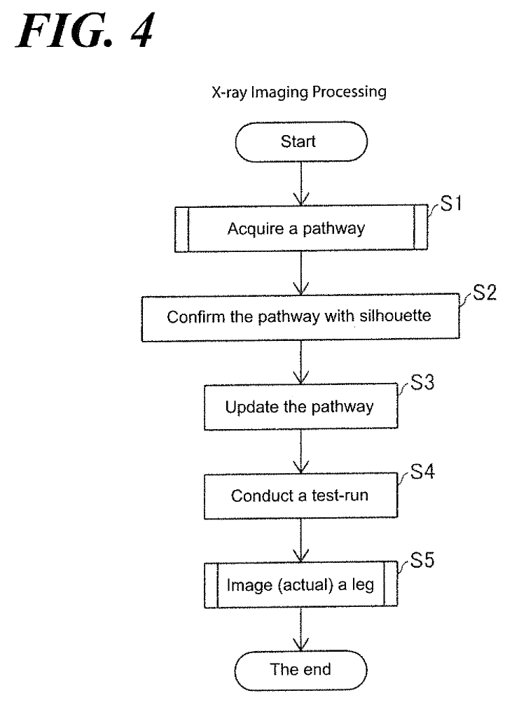

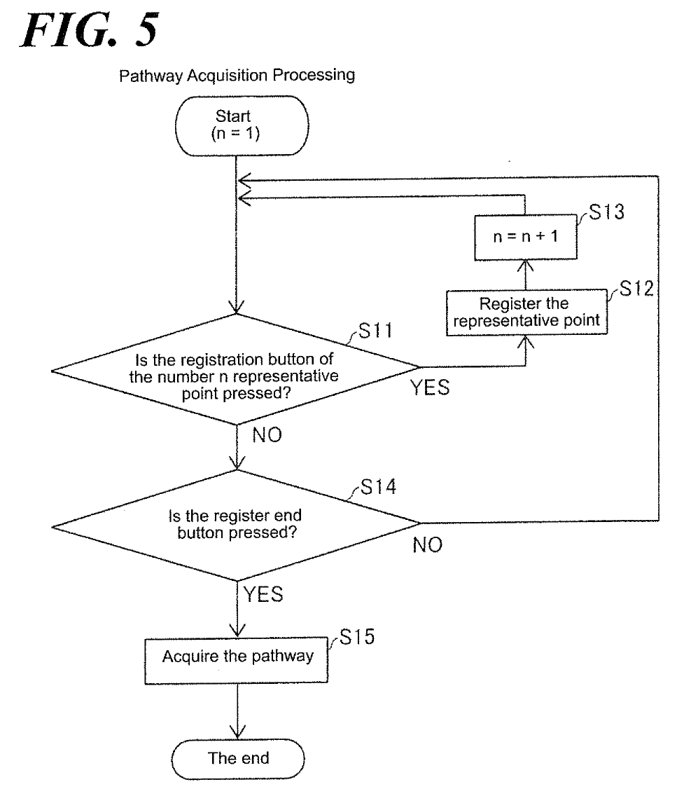

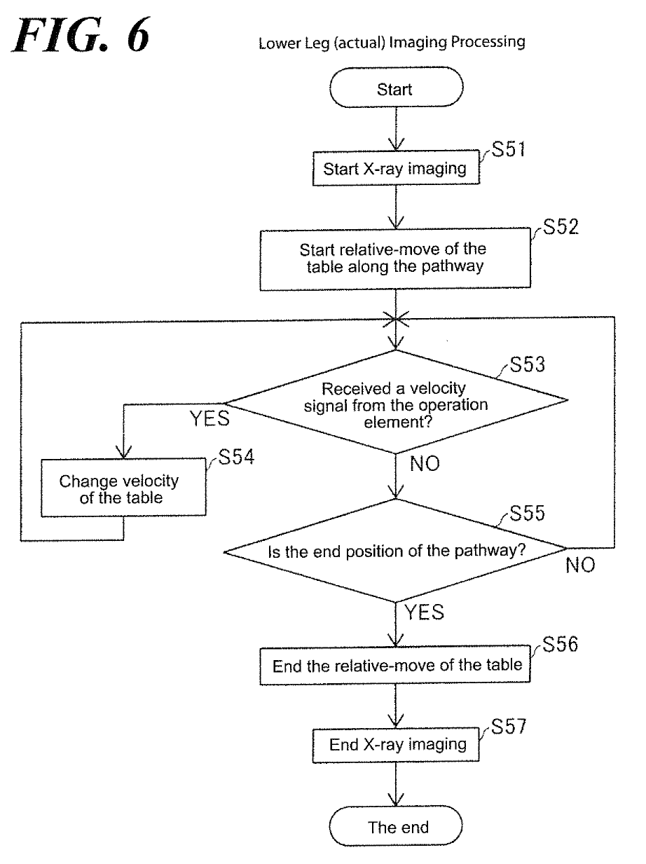

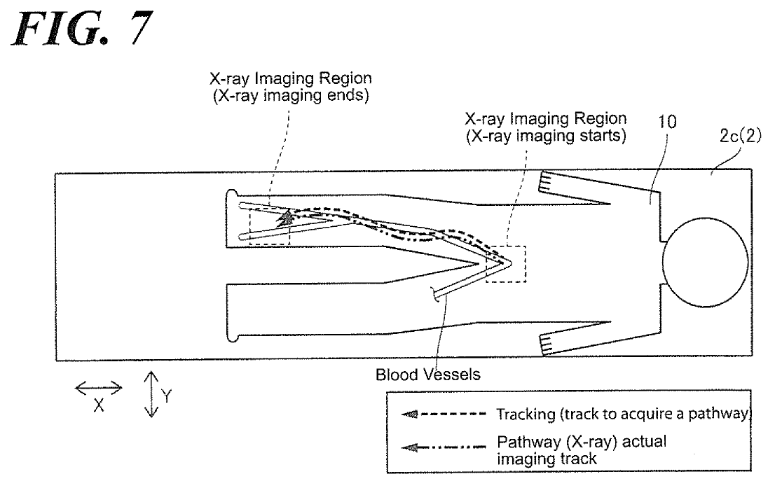

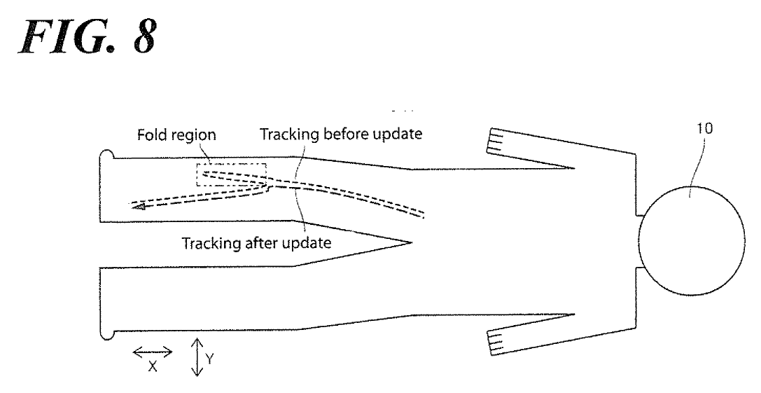

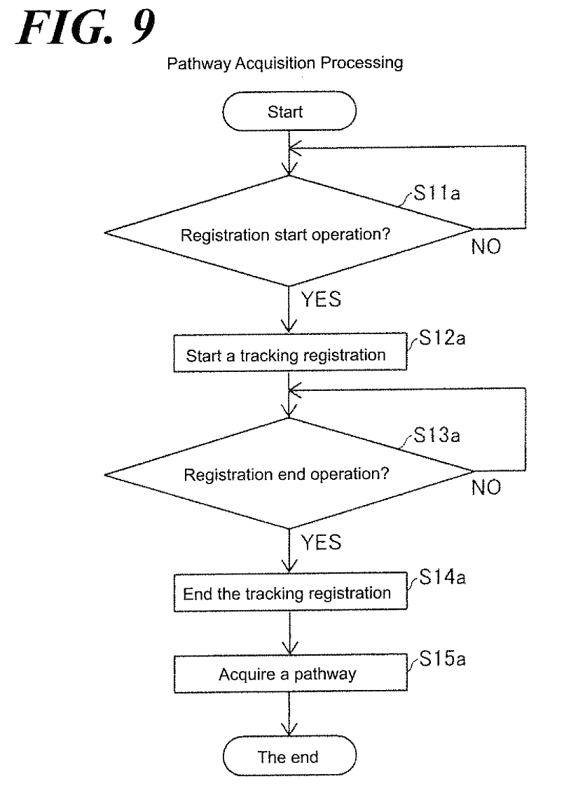

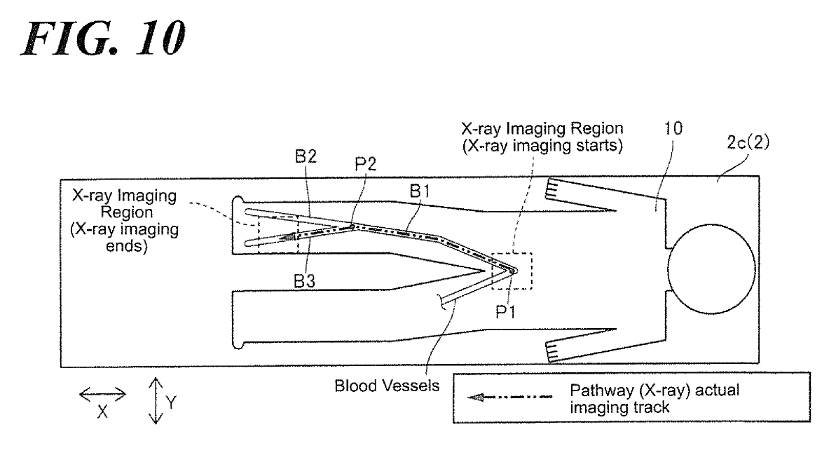

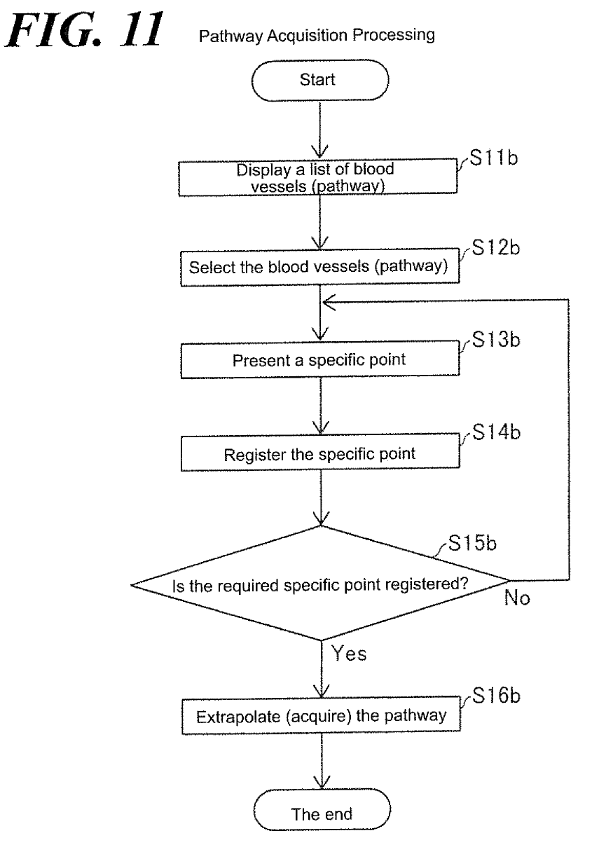

FIG. 1 is a schematic view illustrating an entire structure of an X-ray imaging apparatus according to the aspect of the Embodiment 1 of the present invention.

FIG. 2 is a schematic view illustrating the registration of representative points in order to acquire a pathway.

FIG. 3 is a schematic view illustrating confirmation of a pathway by overlapping the pathway and a silhouette.

FIG. 4 is a flow-chart illustrating an X-ray imaging processing according to the aspect of the Embodiment 1 of the present invention.

FIG. 5 is a flow-chart illustrating a pathway acquisition processing according to the aspect of the Embodiment 1 of the present invention.

FIG. 6 is a flow-chart illustrating an (actual) imaging processing as for a lower leg according to the aspect of the Embodiment 1 of the present invention.

FIG. 7 is a schematic view illustrating the registration of a tracking in order to acquire a pathway.

FIG. 8 is a schematic view illustrating an update of a tracking in order to acquire a pathway.

FIG. 9 is a flow-chart illustrating a pathway acquisition processing according to the aspect of the Embodiment 2 of the present invention.

FIG. 10 is a schematic view illustrating the registration of representative points in order to acquire a pathway and blood vessel (blood pathway.)

FIG. 11 is a flow-chart illustrating a pathway acquisition processing according to the aspect of the Embodiment 3 of the present invention.

### DETAILED DESCRIPTION OF THE PREFERRED EMBODIMENTS

The inventor sets forth specific Embodiments of the present invention based on the following FIGs.

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

It will be further understood by those of skill in the art that the apparatus and devices and the elements herein, without limitation, and including the sub components such as detection elements, X-ray tubes, control elements of all kinds, control elements, drivers, driving elements, display elements, operation elements, inputs, sensors, detectors, memory elements, processors and combinations of these structures etc. will be understood by those of skill in the art as also being identified as or capable of being structures and circuits or combinations of circuits and structures that accomplish the functions without restrictive language or label requirement since those of skill in the art are well versed in computer and operational controls and technologies of radiographic devices and all their sub components, including various circuits and combinations of circuits without departing from the scope and spirit of the present invention.

### Embodiment 1

[System of an X-Ray Imaging Apparatus]

First, referring to FIG. 1 to FIG. 3, the inventor illustrates the system of the X-ray imaging apparatus 100 according to the aspect of the Embodiment 1 of the present invention.

An X-ray imaging apparatus 100 according to the aspect of the Embodiment 1 is structured to image (take an image) blood vessels of such as a lower leg and so forth of a subject (be subject to imaging (human) using a contrast agent. Further, the X-ray imaging apparatus 100 comprises an X-ray irradiation detection element 1, a table 2, a control element 3, an image processing element 4, a display element 5 and an operation element 6. The X-ray irradiation detection element 1 comprises an X-ray tube 1a and a FPD (flat panel detector) 1b. The X-ray tube 1a is connected to a driver 1c. The table 2 is structured to be horizontally movable in the X-direction and Y-direction by a table driving element 2a. The table driving element 2a is connected to the driver 2b. In addition, the table 2 can be movable not only horizontally (in the X-direction and the Y-direction), but also vertically (in the Z-direction). Further, the table can be movable in any two directions of the X-direction, the Y-direction and the Z-direction. For example, the table 2 can be movable in the two directions consisting of the length direction (X-direction) and the vertical direction (X-direction). Further, the X-ray tube 1a is an example of an “X-ray irradiation element” in the Claims. Further, the FPD 1b is an example of an “X-ray detection element” in the Claims. Further, the operation element 6 is an example of a “velocity operation element” in the Claims.

The X-ray imaging apparatus 100 is structured to image the blood vessel of the lower leg of the subject 10 who is lying face up on the table 2. Specifically, the X-ray imaging apparatus 100 is structured to image an X-ray image by receiving the X-ray, irradiated from the X-ray tube 1a arranged below the table 2. transmitting the subject 10 with the FPD 1b. Further, when an imaging is performed, a contrast agent is injected into the blood vessel of the lower leg of the subject 10 using a catheter (not shown in FIG.) and then the X-ray imaging is performed. Accordingly, the blood vessels flow (blood vessels form) can be clearly imaged with the contrast agent through which X-rays hardly pass.

Here, the X-ray imaging apparatus 100 conducts relative-move of the table 2 relative to the X-ray irradiation detection element 1 along the pre-registered pathway by the user (operator) when the actual X-ray imaging is performed. Specifically, according to the X-ray imaging apparatus 100, the registration of the pathway is conducted by the user prior to the actual X-ray imaging. The pathway is a way of the relative-move of the table 2 relative to the X-ray irradiation detection element 1 when an actual imaging is performed. The detail of registration operation will be set forth later. In addition, the relative-move includes any one of cases consisting of the case when the X-ray irradiation detection element 1 (c-arm) is fixed so as not to move and also the table 2 is moved relative to the X-ray irradiation detection element 1, the case when the table 2 is fixed so as not to move and also the X-ray irradiation detection element 1 is moved relative to the table 2, and both X-ray irradiation detection element 1 and table 2 are moved together. Further, the X-ray irradiation detection element 1 can be movable in the X-direction, the Y-direction and the Z-direction and also can be movable in any two directions of the X-direction, the Y-direction and the Z-direction. Further, when either the X-ray irradiation detection element 1 or the table 2 is moved, the relative position of the X-ray irradiation detection element 1 and the table 2 (the irradiation position of the X-ray toward the subject) is used as a pathway data described later. Further, the move object can be different between the time on the registration of the pathway data and the time on the operation (on the imaging). For example, when the pathway is registered, the table 2 is moved without moving the X-ray irradiation detection element 1, and when the operation (imaging) is performed, the X-ray irradiation detection element 1 can be moved without moving the table 2. Further, at least one of the X-ray irradiation detection element 1 and the table 2 can be movable in the X-direction, the Y-direction and the Z-direction, so that even if the X-ray irradiation detection element (C-arm) 1 is angled (even if tilted), the pathway of the blood vessels can be tracked 3-dimensionally by the X-ray irradiation detection element on the imaging. Further, the X-ray irradiation detection element 1 can be moved in the vertical direction by the up-and-down moving mechanism (not shown in FIG.) that is installed to the X-ray irradiation detection element 1. Further, the table 2 can be moved in the vertical direction by the up-and-down moving mechanism (not shown in FIG.) that is installed to the table 2.

Referring to FIG. 1, the X-ray tube 1a is supported by the C-type arm 1d. Specifically, the X-ray tube 1a is supported by the C-type arm 1d facing the FPD 1b as sandwiching the table 2. Further, the X-ray tube 1a irradiates X-rays to the subject 10 when the pathway is registered and the actual X-ray imaging is performed. Specifically, the X-ray tube 1a irradiates (fluoroscopes) a weak X-ray (lower X-ray dose than a specified value) and irradiates stronger X-rays than fluoroscopy thereof when the X-ray (actual) imaging is performed. Further, the X-ray tube 1a is driven by the driver 1c to irradiate an X-ray. The driver 1c is connected to the control element 3. Further, the X-ray tube 1a can be adjusted as to the intensity of generated X-rays and the irradiation area.

Referring to FIG. 1, the FPD 1b is supported by the C-type arm 1d. Further, the FPD 1b detects the X-ray irradiated from the X-ray tube 1a and then transmitting the subject 10 and detects an X-ray image. Further, the FPD 1b images the X-ray image based on the X-ray. Specifically, the FPD 1b converts the detected X-ray to an electric signal. Further, the X-ray data converted to the electric signal is sent to the image processing element 4.

The X-ray irradiation detection element 1 (X-ray tube 1a and FPD 1b) starts imaging an X-ray image based on the instruction for starting imaging input from a user (an operator) through the operation element 6. Specifically, the X-ray is irradiated from the X-ray tube 1a and detected by the FPD 1b based on the instruction for staring imaging from the user. The X-ray irradiation detection element 1 (X-ray tube 1a and FPD 1b) starts imaging an X-ray image based on the instruction for starting imaging input from a user (an operator) through the operation element 6. Specifically, the X-ray irradiation from the X-ray tube 1a is suspended and detection by the FPD 1b is suspended based on the instruction for ending or holding imaging from the user.

Referring to FIG. 1, the table 2 includes a loading plane 2c on which the subject lies. Further, referring to FIG. 1, the table 2 is relatively movable to the X-ray irradiation detection element 1 (X-ray tube 1a and FPD 1b) under the state in which the subject 10 is placed (lying) on the loading plane 2c. Specifically, the table 2 is movable in the length direction (X-direction) corresponding to the body axis of the lying subject 10 and in the width direction (Y-direction) corresponding to the side direction of the subject 10. The length direction and width direction are orthogonal each other in the loading plane 2c. The table driving element 2a is connected to the control element 3 through the driver 2b. The table driving element 2a drives the table 2 under a control of the control element 3. Further, when the table 2 is driven by the table driving element 2a, the table 2 can be driven according to the user's operation. Further, the table 2 can be also manually moved by the used in the length direction and width direction. Further, the length direction is an example of “first direction of the Claims. Further, the width direction is an example of “second direction of the Claims.

The control element 3 controls each element of the X-ray imaging apparatus 100. Specifically, the control element 3 controls the X-ray tube 1a through the driver 1c. Further, the control element 3 controls drive of the table driving element 2a through the driver 2b. Further, the control element 3 controls the display element 5 so as to display a real-time image when the subject is fluoroscoped upon the registration operation of the pathway. Further, the control element 3 controls the display element 5 so as to display an X-ray image generated by the image processing element 4. Further, the control element 3 receives the operation through the operation element 6. Further, the control element 3 instructs the memory 3a to store the X-ray image.

The control element 3 acquires the pathway based on a positional data of the length direction (X-direction) and width direction (Y-direction) of the table 2 designated by the user relative to the X-ray irradiation detection element 1. Further. the control element 3 moves the table 2 in the length direction (X-direction) and the width direction (Y-direction) relatively to the X-ray irradiation detection element 1 along the pathway (pathway of the relative position between the pre-registered loading plane and the X-ray irradiation detection element 1) based on the pre-registered pathway data including the pathway for the relative-move of the table 2 when the subject is imaged with X-ray by the X-ray irradiation detection element 1. Meanwhile, the pathway data is the data being pre-registered by the registration operation conducted prior to the actual X-ray imaging of the subject 10. Further, the pathway data (pathway) is acquired based on the positional data registered by the registration operation conducted prior to the actual X-ray imaging of the subject 10 under the control of the control element 3 and stored in the memory 3a.

Specifically, a user's operation to determine the pathway during actual X-ray imaging (on imaging) (an operation to determine which pathway is used to move the table 2 in the length direction (X-direction) and width direction (Y-direction) relative to the X-ray irradiation detection element 1) is not mandatory and Further, the control element 3 is capable of controlling the velocity of the relative-move of the table 2 in the length direction (X-direction) and width direction (Y-direction) relative to the X-ray irradiation detection element 1 based on the operation of the operation element 6 during the actual the X-ray imaging (on imaging.) Specifically, the user can relatively move the table 2 relative to the X-ray irradiation detection element 1 while changing the velocity of the relative-move of the table 2 relative to the X-ray irradiation detection element 1 during the actual imaging (on imaging).

Here, referring to FIG. 2, the above described positional data should include data of plural representative points D1 to D4 of the table 2 designated by the user's registration operation relative to the X-ray irradiation detection element (referring to FIG. 1.) The plural representative points D1 to D4 of the table 2 are plural points on the pathway where the table 2 is positioned relative to the X-ray irradiation detection element 1 so that the X-ray can be irradiated to the subject 10, and the plural points indicating the center position of the X-ray irradiation area from the output direction of the X-ray. And referring to FIG. 1, the control element 3 acquires (generates) the pathway based on the data of plural representative points D1 to D4 of the table 2. Specifically, the control element 3 acquires the pathway by interpolating plural representative points D1 to D4 of the table 2 with a straight line designated by the user's registration operation In addition, the control element 3 specifies the designated representative points in order as near the start point of the pathway. Specifically, the control element 3 conducts X-ray imaging in order (D1, D2, D3 and D4 in order) in first designated representative points upon the actual X-ray imaging.

The control element 3 conducts a test-run to make sure that the relatively moving X-ray irradiation detection element 1 and the table 2 are not going to interfere each other when the registration operation is conducted. Further, the control element 3 starts the test run from the ending position of the registration operation and ends the test-run at the start point of the registration operation. Specifically, the control element 3 conducts the test-run in the reverse tracking of the tracking of relative-move of the table 2 relative to the X-ray irradiation detection element 1 upon the registration operation. For example, when the registration operation starts from the right groin and ends at the right toe, the control element 3 starts the test-run from the right toe and ends the test-run at the right groin. As results, when the test-run ends, the position of the X-ray irradiation detection element 1 relative to the table 2 becomes the position of the start position of the X-ray imaging, so that the position of the X-ray irradiation detection element 1 relative to the table 2 is not required to be moved from the end position of the registration operation only for starting the X-ray imaging. Accordingly, the time needed for an X-ray imaging can be shorted.

Referring to FIG. 3, the control element 3 overlaps the schematic silhouette of the subject 10 (refer to FIG. 1) and the acquired pathway and displays on the display element 5 (refer to FIG. 1) in order to make sure the general position of the pathway when the registration operation is conducted. The control element 3 overlaps the schematic silhouette of the subject 10 (refer to FIG. 1) and the acquired pathway and in addition, overlaps a long image (e.g., an image of blood vessels) obtained by the fluoroscopy thereon and displays on the display element 5 (refer to FIG. 1) in order to make sure the general position of the pathway when the fluoroscopy is performed (X-ray irradiation) upon the registration operation.

The control element 3 is capable of updating the pathway based on the revision instruction from the user through the operation element 6 following the registration operation. In detail, the control element 3 displays the pathway on the display element 5 when the pathway has been determined by the registration operation. And when the position of the representative point on the pathway displayed on the display element 5 is moved through the operation element 6, the control element 3 updates the pathway so that the pathway can pass the representative point after moving. A method to move the position of the representative point can be the method of using a mouse (operation element 6) to move or a touch panel (the operation element 6 and the display element 5.) Further, as to updating, an addition of a representative point other than moving the representative point can be applied.

The image processing element 4 generates the digital X-ray image based on the received X-ray data sent from the FPD 1b. The data of the generated digital X-ray image is acquired by the control element 3 and displayed on the display element 5.

The display element 5 displays a real-time image when the subject is fluoroscoped upon the registration operation of the pathway. Further, the display element 5 displays an acquired pathway based on the registration operation by the user. Further, the display element 5 displays an X-ray image that is imaged and acquired upon the actual X-ray imaging.

The operation element 6 is capable of receiving a variety of user operations including instruction related to starting an X-ray imaging, ending the X-ray imaging, staring the registration operation, ending the registration operation and so forth. For example, the operation element 6 comprises a registration switch to set the designation of the representative point and a registration switch to suspend the registration operation of the representative point. Further, the operation element 6 is capable of receiving a user operation controlling the velocity of the relative-move of the table 2 along the pathway in the length direction (X-direction) and width direction (Y-direction) relative to the X-ray irradiation detection element 1 during the actual the X-ray imaging (on imaging.) For example, the operation element 6 is capable of selecting one velocity level out of three velocity levels including “fast”, ‘slow” and “hold”. When the velocity level is selected, the operation element 6 sends the velocity signal to the control element 3. In addition, as for the operation element 6, a keyboard, a mouse, a touch panel, or a foot switch operable by a user's foot and so forth can be applied.

(X-Ray Imaging Processing)

Next, referring to FIG. 4, the inventor sets forth an X-ray imaging processing by the X-ray imaging apparatus 100 according to the aspect of the Embodiment 1.

At the step S1, the pathway allows the table 2 to relative-move relative to the X-ray irradiation detection element 1 by the registration operation. The detail of the step S1 will be set forth later.

At the step S2, the pathway is confirmed by the silhouette S. Specifically, an image in which the schematic silhouette S of the subject 10 (refer to FIG. 3) and the acquired pathway are overlapped is displayed on the display element 5 in order to make sure the position of the pathway.

At the step S3, the pathway is revised and updated. Specifically, the acquired pathway is displayed on the display element 5. And when the position of the representative point is moved through the operation by the user with the mouse and so forth, the control element 3 updates the pathway so that the pathway can pass the representative point after moving.

At the step S4, a test-run is conducted. Specifically, when the registration operation starts from the right groin and ends at the right toe, the test-run starts from the right toe and ends at the right groin.

At the step S5, the X-ray imaging of the lower leg is performed by allowing the table 2 to relative-move relative to the X-ray irradiation detection element 1 along the acquired pathway. The detail of the step S5 will be set forth later.

(Pathway Acquisition Processing)

Next, referring to FIG. 5, the inventor sets forth a pathway acquisition processing (the sub-routine of the step S1) based on the registration operation by the X-ray imaging apparatus 100 according to the aspect of the Embodiment 1.

At the step S1, it is determined whether the first (number n) representative point is registered or not. Specifically, it is determined whether the registration switch of the operation element 6 is pushed or not under the state in which the table 2 is in place at the position of the first (number n) representative point relative to the X-ray irradiation detection element 1. If the registration switch is pushed, the step proceeds to the step S12 and the number n representative points are registered. Then at the step S13, 1 is added to n and the step returns to S11. In addition, if the representative point is just 1, the pathway cannot be obtained (the distance between two representative points cannot be interpolated by a line segment), so that the step S11 to the step S13 are repeated multiple times (at least twice.) Further. if the registration switch is not pushed, the step proceeds to the step S14.

At the step S14, it is determined whether the registration end switch of the operation element 6 is pushed or not. If the registration end switch is not pushed, the step returns to the step S11 and if the switch is pushed, the step proceeds to the step S15.

At the step S15, the distance between plural representative points is interpolated by the straight line, so that the pathway is acquired and then the pathway acquisition processing is completed.

(Lower Leg (Actual) Imaging Processing)

Next, referring to FIG. 6, the inventor sets forth a lower leg (actual) imaging processing (the sub-routine of the step S5) based on the registration operation by the X-ray imaging apparatus 100 according to the aspect of the Embodiment 1.

At the step S51, an X-ray imaging starts by the X-ray irradiation detection element 1. Specifically, an X-ray is irradiated from the X-ray tube 1a to the subject 10 and the FPD 1b detects the transmitting X-ray through the subject 10.

At the step S51, the relative-move of the table 2 relative to the X-ray irradiation detection element 1 starts along the pathway. For example, the relative-move of the table 2 relative to the X-ray irradiation detection element 1 starts from the right groin to the right toe.

At the step 53, it is determined whether the control element 3 received a velocity signal from the operation element 6 or not. And if the control element 3 has received a velocity signal from the operation element 6, the step proceeds to the step S54. For example, in the case of that the setting of the velocity of the relative-move of the table 2 relative to the X-ray irradiation detection element 1 is “slow”, when the control element 3 receives the velocity signal so as to change the setting to “fast” from the operation element 6, the step proceeds to the step S54. At the step S54, the velocity of the relative-move of the table 2 relative to the X-ray irradiation detection element 1 is changed and the step returns to the step S53. Further, at the step S53, if the control element 3 has not received the velocity signal from the operation element 6, the step proceeds to the step S55.

At the step S55, it is determined that the position of the table 2 relative to the X-ray irradiation detection element 1 (the position of the X-ray irradiation region) has been end position of the pathway or not. If the position has not yet been the end position, the step returns to the step S53, but if the position has been the end position of the pathway, the step proceeds to the step S56.

At the step S56, the relative-move of the table 2 relative to the X-ray irradiation detection element 1 ends. And the lower leg (actual) imaging processing ends at the step S5. Specifically, the irradiation from the X-ray tube 1a is suspended and the lower leg (actual) imaging processing ends.

Effect According to the Aspect of the Embodiment 1

The following effects can be obtained according to the aspect of the Embodiment 1.

According to the aspect of the Embodiment 1, as set forth above, when the X-ray imaging of the subject 10 is performed, the table 2 is moved by the control element 3 in the length direction and the width direction orthogonal each other in the loading plane 2c along the pathway of the relative position between the pre-registered loading plane 2c and the X-ray irradiation detection element 1. Accordingly, the control element 3 moves automatically the table relatively in the length direction and the width direction relative to the X-ray irradiation detection element along the pre-registered pathway, so that the user's operational workload can be reduced when an X-ray imaging is performed while relatively moving the table 2 in the 2 axes directions (the length direction and the width direction) in the loading plane 2c relative to the X-ray radiation detection element 1. As results, even when the X-ray imaging area is narrowed to suppress the X-ray dose being irradiated to the subject 10, the imaging target region of the subject 10 can be automatically followed and the imaging target region of the subject 10 can be imaged easily and adequately.

Further, according to the aspect of the Embodiment 1 as described above, the operation element 6 is installed to receive the operation to control the velocity of the relative-move of the table 2 along the pathway in the length direction and width direction relative to the X-ray irradiation detection element 1, and the control element 3 controls the velocity of the relative-move of the table 2 in the length direction and width direction relative to the X-ray irradiation detection element 1 based on the operation of the operation element 6. Accordingly, the user should control only the velocity of the relative-move of the table moving in the length direction and width direction relative to the X-ray irradiation detection element, so that the user can facilitate to track the contrast agent moving in blood vessels when an X-ray imaging of blood vessels is performed using the contrast agent. As results, when the X-ray imaging is performed while moving the table 2 in the 2 axes directions in the loading plane 2c relative to the X-ray irradiation detection element 1, the operation workload of the user can be reduced further.

Further, according to the aspect of the Embodiment 1 as described above, the control element 3 acquires the pathway based on a positional data of the length direction and the width direction of the table 2 designated by the user relative to the X-ray irradiation detection element 1. Accordingly. the user can set the pathway every X-ray imaging. a necessary X-ray image of the subject 10 can be acquired adequately.

Further, according to the aspect of the Embodiment 1 as described above, the positional data should include data of plural representative points of the table 2 designated by the user's operation relative to the X-ray irradiation detection element 1, and the control element 3 acquires the pathway based on the data of plural representative points of the table 2. Accordingly, the pathway is acquired based on the representative points, so that the user can easily to set up the pathway by designating the representative points along the imaging target region of the subject 10.

Further, according to the aspect of the Embodiment 1 as described above, the control element 3 acquires the pathway by interpolating with the straight line or the curved line between plural representative points. Accordingly, the user can set up easily the pathway by interpolating without designating many representative points.

Further, according to the aspect of the Embodiment 1 as described above, the X-ray imaging apparatus 100 comprises the display element 5 to display the pathway and the control element 3 can update the pathway displayed on the display element based on the revision instruction from the user. Accordingly, the display element 5 can display the target pathway to be updated. so that the user can easily revise the tracking while watching the display element 5 and confirming the display.

### Embodiment 2

Next, referring to FIG. 1, FIG. 7 to FIG. 9, the inventor sets forth an operation of the X-ray imaging apparatus 200 according to the aspect of the Embodiment 2. The aspect of the Embodiment 2 is different from the aspect of the Embodiment 1, in which the pathway is acquired by the data of plural representative points designated by the user, but according to the aspect of the Embodiment 2, the pathway is acquired based on the trace data of the table 2 relatively moved relative to the X-ray irradiation detection element 1 by the user operation.

Referring to FIG. 1, an X-ray imaging apparatus 200 according to the aspect of the Embodiment 2 images (takes an image) blood vessels of such as a lower leg and so forth of a subject 10 (be subject to imaging (human) using a contrast agent. Further, the X-ray imaging apparatus 200 comprises an X-ray irradiation detection element 1, a table 2, a control element 31, an image processing element 4, a display element 5 and an operation element 6.

Referring to FIG. 7, the control element 31 acquires the pathway based on a positional data of the length direction (X-direction) and width direction (Y-direction) of the table 2 designated by the user relative to the X-ray irradiation detection element 1 (referring to FIG. 1.) The positional data includes a tracking data of the relative-move of the table 2 relative to the X-ray irradiation detection element 1 instructed by the user's registration operation. And the control element 31 (referring to FIG. 1) acquires (generates) the pathway based on the tracking data of the table 2. Here, the tracking is the tracking of the relative-move of the table 2 relative to the X-ray irradiation detection element 1 by the user's manual operation in order to acquire a pathway in the predetermined duration (start to the end of registration operation). And the control element 31 acquires the same pathway as the tracking. However, if the control element 31 cannot get the same pathway as the tracking due to apparatus restrictions (e.g., interference from other equipment), a similar pathway to the tracking is acquired.

The control element 31 registers the tacking along one direction, the length direction (X-direction) of the table 2 Specifically, referring to FIG. 8, the control element 31 registers the tracking from the head side to the toe side of the subject 10 or from the toe side to the head side of the subject 10 upon the tracking registration. Further, the control element 31 re-registers the tracking from the returned position excluding the tracking of the returned portion of the table 2, when the tracking of the table 2 is returned to the other direction upon the tracking registration. Specifically, the control element 31 acquires the pathway based on the tracking after updating revision, excluding the fold region of the tracking before updating (inside rectangular region indicated by the chain double-dashed line.) In addition, referring to FIG. 8, for convenience of explanation, the trackings before and after are not overlapped, but actually the trackings before and after should be overlapped except the fold region.

(Pathway Acquisition Processing)

Referring to FIG. 9, the inventor sets forth a pathway acquisition processing (the sub-routine of the step S1 referring to FIG. 4) based on the registration operation by the X-ray imaging apparatus 200 according to the aspect of the Embodiment 2.

At the step S11a, it is determined whether an operation to register the tracking has started or not. For example, it is determined whether the registration start button on the operation element 6 is pressed or not to start the tracking registration by the user. In addition, when the tracking registration is executed together with the fluoroscopy, the tracking registration can be started by pressing the start button for the fluoroscopy. When the operation to start the tracking registration is not executed (the tracking registration button is not pressed), the step S11a is repeated, but when the operation to start the tracking registration is executed (the tracking registration button is pressed), the step proceeds to the step S12a.

Then at the step S12a, the tracking registration starts. Specifically, the tracking of the relative-move of the table 2 relative to the X-ray irradiation detection element 1 is stored in the memory 3a by the user. In addition, following that the tracking registration button is pressed, the user moves relatively the table 2 relative to the X-ray irradiation detection element 1 in the length direction (X-direction) and width direction (Y-direction) to obtain the tracking. For example, the registration starts from the groin and the table 2 is relatively moved toward the right toe along the lower right leg.

At the step S13a, it is determined whether an operation to end the tracking registration has been executed or not. For example, it is determined whether the registration end button on the operation element 6 is pressed or not to end the tracking registration by the user. In addition, when the tracking registration is executed together with the fluoroscopy, the tracking registration can be ended by pressing the end button for the fluoroscopy. When the operation to end the tracking registration is not executed (the registration end button is not pressed), the step S13a is repeated, but when the operation to end the tracking registration is executed (the registration end button is pressed), the step proceeds to the step S15a. Then at the step S15, the pathway is acquired along the tracking and then the pathway acquisition processing is completed.

In addition, other structure according to the aspect of the Embodiment 2 is the same as the aspect of the Embodiment 1.

Effect According to the Aspect of the Embodiment 2

The following effects can be obtained according to the aspect of the Embodiment 2.

According to the aspect of the Embodiment 2, as well as the aspect of the Embodiment 1 as described above, the control element 31 moves the table 2 relatively in the length direction and the width direction, orthogonal in the loading plane 2c, relative to the X-ray irradiation detection element 1 along the pathway based on the pre-registered pathway data including the pathway for the relative-move of the table 2 when the subject 10 is imaged with X-ray. As results, when the X-ray imaging is performed while moving the table 2 in the 2 axes directions (length direction and width direction) in the loading plane 2c relative to the X-ray irradiation detection element 1, the operation workload of the user can be reduced further.

In addition, according to the aspect of the Embodiment 2, the positional data includes a tracking data of the relative-move of the table 2 relative to the X-ray irradiation detection element 1 instructed by the user's operation, and the control element 31 acquires the pathway based on the tracking data of the table 2. Accordingly, the pathway can be acquired by the user's operation based on the tracking data of the table 2 relatively moved relative to the X-ray irradiation detection element 1, so that the pathway can be set by relatively easy operation.

In addition, according to the aspect of the Embodiment 2 as describe above, the control element 31 registers the tracking along one direction of the length direction of the table 2 and when the tracking of the table 2 is returned to the other direction, the tracking is re-registered from the returned position excluding the tracking of the returned portion of the table 2. Accordingly, the tracking can be updated by just returning the table, so that the tracking can be easily updated.

In addition, other effects according to the aspect of the Embodiment 2 are the same as the aspect of the Embodiment 1.

### Embodiment 3

Next, referring to FIG. 1, FIG. 10 and FIG. 11, the inventor sets forth an operation of the X-ray imaging apparatus 300 according to the aspect of the Embodiment 3. The inventors set forth the aspect of the Embodiment 3 is different from the aspect of the Embodiment 1, in which the pathway is acquired by only the data of plural representative points designated by the user, but according to the aspect of the Embodiment 3, the pathway is presumed (acquired) based on the data identifying blood vessels and the data of the specific point identifying a blood pathway.

Referring to FIG. 1, an X-ray imaging apparatus 300 according to the aspect of the Embodiment 3 images (takes an image) blood vessels of such as a lower leg and so forth of a subject 10 (be subject to imaging (human) using a contrast agent, Further, the X-ray imaging apparatus 300 comprises an X-ray irradiation detection element 1, a table 2, a control element 32, an image processing element 4, a display element 5 and an operation element 6.

The control element 32 presumes (acquires) the pathway based on a positional data of the length direction (X-direction) and width direction (Y-direction) of the table 2 designated by the user relative to the X-ray irradiation detection element 1. The positional data includes specific points of a subject 10. The specific point is the bifurcation or turning of blood vessels (blood vessel pathway), which common specific points are found at almost the same position of each subject. Further, the positional data includes the data identifying blood vessels (blood vessel pathway). Accordingly, the control element 32 presumes (acquires) the pathway based on the data and the specific points identifying the blood vessels (blood vessel pathway) selected by the user. In addition, the data that are identifying of the specific points and the blood vessels (blood vessel pathway)) are stored in a memory 3a.

(Pathway Acquisition Processing)

Next, referring to FIG. 11, the inventor sets forth a pathway acquisition processing (the sub-routine of the step S1 referring to FIG. 4) based on the registration operation by the X-ray imaging apparatus 300 according to the aspect of the Embodiment 3.

At the step S11b, a list of blood vessels (blood vessel pathway) as the data identifying the blood vessels (blood vessel pathway) is displayed on the display element 5 is displayed (display.) For example, referring to FIG. 10, the blood vessel Bi extends from the groin to near the knee and bifurcates from the blood vessel B1 to blood vessels B2 and B3 extending to each toe which are displayed (presented) on the display element 5. In addition, the display format of blood vessels (presentation format to the user) is not limited to the figurative format referring to FIG. 10, but only each blood vessel (the name of blood vessel) can be listed up.

At the step S12b, a predetermined blood vessel among the blood vessels (the name of blood vessel) can be selected among the displayed list of the blood vessels (the name of blood vessel.) For example, the blood vessel B1 and the blood vessel B2 shall be selected.

At the step S13b, necessary specific points are displayed (presented) to presume the more exact position of the selected blood vessel. For example, the groin side end point of the blood vessel B1 is displayed as the specific point P1 and the instruction, “Press the registration button at the specific point”, is displayed on the display element 5. And at the step S14b, the table 2 is relatively moved relative to the X-ray irradiation detection element 1 to the position of the specific point and the registration button on the operation element 6 to register the specified point is pressed.

At the step S15b, it is determined whether the specific point that is required to presume (acquire) the pathway is registered or not. Specifically, if the specific point to be further registered to presume the pathway (e.g., a point (bifurcation) P2 connecting blood vessels B1 and blood vessels B3) exists, the step S13b and the step 14b are repeated and once all required specific points are registered, the step proceeds to the step S16.

At the step S16b, the pathway is presumed (acquired) based on the data of the registered blood vessels (blood vessel pathway) and the specific points and the pathway acquisition processing is completed.

In addition, other structure according to the aspect of the Embodiment 3 is the same as the aspect of the Embodiment 1.

Effect According to the Aspect of the Embodiment 3

The following effects can be obtained according to the aspect of the Embodiment 3.

According to the aspect of the Embodiment 3, as well as the aspect of the Embodiment 1 as described above, the control element 32 moves the table 2 relatively in the length direction and the width direction, orthogonal in the loading plane 2c, relative to the X-ray irradiation detection element 1 along the pathway based on the pre-registered pathway data including the pathway for the relative-move of the table 2 when the subject 10 is imaged with X-ray. As results, when the X-ray imaging is performed while moving the table 2 in the 2 axes directions (length direction and width direction) in the loading plane 2c relative to the X-ray irradiation detection element 1, the operation workload of the user can be reduced further.

In addition, according to the aspect of the Embodiment 3 as described above, the positional data includes data identifying the blood vessels and the control element 32 acquires the pathway based on the identified blood vessels. Accordingly, the pathway can be acquired along the identified blood vessel common to each subject 10, so that the pathway can be further easily acquired.

In addition, according to the aspect of the Embodiment 3 as described above, the positional data includes the data identifying the blood vessels and the control element 32 extrapolate the pathway based on the data of the specific points in addition to the data identifying the blood vessels. Accordingly, the pathway can be extrapolated based on the data of specific points in addition to the data identifying the blood vessels, so that the pathway can be easily set up regardless an experience level of the user.

In addition, other effects according to the aspect of the Embodiment 3 are the same as the aspect of the Embodiment 1.

### Alternative Embodiment

In addition, the aspects of the Embodiments disclosed at this time are examples and not limited thereto in any points. The scope of the present invention is specified in the claims but not in the above description of the aspect of the Embodiments and all alternative (alternative examples) are included in the scope of the claims and equivalents thereof.

For example, according to the aspect of the Embodiments 1 to 3 described above, the example of that the lower leg of the subject is imaged by the X-ray is described but the present invention is not limited thereto. According to the present invention, an arm, an abdomen, other regions than the lower leg of the subject can be subject to an X-ray imaging.

In addition, according to the aspect of the Embodiments described above, the example of that the lying subject is imaged by the X-ray is described but the present invention is not limited thereto. According to the present invention, a standing subject (standing posture) can be imaged.

In addition, according to the aspect of the Embodiments 1 to 3 described above, the example of that an X-ray is irradiated from the X-ray tube upon the registration operation of the pathway is described, but the present invention is not limited thereto. According to the present invention, the X-ray irradiation detection element can further include a lighting device (not shown in FIG.) that irradiates the light to the subject without irradiating an X-ray from the X-ray tube upon the registration operation of the pathway. Accordingly, the imaging area can be identified without an X-ray irradiation.

In addition, according to the aspect of the Embodiments 1 to 3 described above, the example of that the test-run is conducted during an X-ray imaging processing, but the present invention is not limited thereto. According to the present invention, the test-run during the X-ray imaging processing is not mandatory.

In addition, according to the aspect of the Embodiments 1 described above, the example of that the distance between the representative points is interpolated by the straight line is described but the present invention is not limited thereto. According to the present invention, the curve line other than the straight line can be applied to interpolate the distance between representative points.

In addition, according to the aspect of the Embodiments 3 described above, the example of that the pathway is extrapolated (acquired) based on the data of the blood vessels (pathway) of the subject, but the present invention is not limited thereto. According to the present invention, the pathway can be extrapolated based on the data of bones and organs of the subject.

In addition, according to the aspect of the Embodiments 1 to 3 described above, for convenience of explanation, the inventors set forth a processing of the X-ray imaging apparatus according to the present invention is processed in order following the process flow using the flow driving flow chart, but the present invention is not limited thereto. According to the present invention, the processing operation can be performed using an invent driving processing (invent driven processing) every event. In such case, a perfect event driven processing can be applied or a combination of the event driven processing and flow driven processing can be applied.

Further, according to the aspect of the Embodiments 1 to 3 described above, the example of the aspect, in which one X-ray irradiation detection element 1 is installed (single plane), is illustrated but the present invention is not limited thereto. According to the present invention, two X-ray irradiation detection elements can be installed (bi-planes). In addition, when two X-ray irradiation detection elements are installed, at least one of the X-ray irradiation detection element and the table can be movable in the X-direction, the Y-direction and the Z-direction, so that the pathway of the blood vessels can be tracked 3-dimensionally by the X-ray irradiation detection element on the imaging.

### REFERENCE OF SIGNS

1 X-ray irradiation detection element
1a X-ray tube (X-ray irradiation element)
1b FPD (X-ray detection element)
2 Table
2c Loading plane
3, 31, 32 Control element
5 Display element
6 Operation element (Velocity operation element)
10 Subject
100 Mobile X-ray imaging apparatus

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An X-ray imaging apparatus, comprising:
an X-ray irradiation detection element including an X-ray irradiation element that irradiates an X-ray to a subject and an X-ray detection element that detects the X-ray transmitting said subject;
a table that can load the subject on a loading plane; and
a control element that moves at least one of said X-ray irradiation detection element and a first side of said table in one of a first direction and a second direction, said second direction orthogonal to said first direction, along a pathway of a relative position of a pre-registered loading plane and said X-ray irradiation detection element when an X-ray imaging of the subject is performed.

2. The X-ray imaging apparatus, according to claim 1, further comprising:
a velocity operation element that receives an operation instruction to control a velocity of a relative-move of said table moving along said pathway in said first direction and said second direction relative to said X-ray irradiation detection element; and
wherein said control element operatively controls a velocity of the relative-move of said table moving in said first direction and said second direction relative to said X-ray irradiation detection element based upon the operation of said velocity operation element.

3. The X-ray imaging apparatus, according to claim 2, wherein:
said control element is a circuit and acquires said pathway based upon a positional data of said first direction and said second direction of said table designated by a user relative to the X-ray irradiation detection element.

4. The X-ray imaging apparatus, according to claim 3, wherein:
said positional data includes data of plural representative points of said table designated by the user's operation relative to said X-ray irradiation detection element, and said control element acquires said pathway based upon the data positions of plural representative points of said table.

5. The X-ray imaging apparatus according to claim 4, wherein:
said control element is a circuit and acquires said pathway by interpolating the distance between said plural representative points with a straight line or a curved line.

6. The X-ray imaging apparatus according to claim 5, wherein:
said positional data further includes a tracking data of a relative-move of said table relative to said X-ray irradiation detection element; and
said control element acquires said pathway based on the tracking data of said table.

7. The X-ray imaging apparatus according to claim 6, wherein:
said positional data further includes data identifying blood vessels; and
said control element acquires said pathway based on said identified blood vessels.

8. The X-ray imaging apparatus according to claim 7, wherein:
said positional data further includes data of a specific point to extrapolate the pathway of said blood vessel; and
said control element extrapolates said pathway based on the data of said specific point in addition to the data identifying said blood vessels.

9. The X-ray imaging apparatus according to claim 6, wherein:
said control element registers said tracking along one direction of the length direction of said table, and when the tracking of said table is returned to the other direction, the tracking is re-registered from the returned position excluding the tracking of the returned portion of said table.

10. The X-ray imaging apparatus according to claim 9, further comprising:
a display element that visually displays said pathway; and
wherein said control element is capable of updating said pathway displayed on said display element based on a revision instruction from a user.
